(19) United States
(12) Patent Application Publication
(10) Pub. No.: US 2019/0151825 A1
(43) Pub. Date: May 23, 2019

(54) METAL/SUPPORT CATALYST FOR CONVERSION OF CARBON DIOXIDE TO METHANE

(71) Applicant: KOREA INSTITUTE OF SCIENCE AND TECHNOLOGY, Seoul (KR)

(72) Inventors: Hyoungchul KIM, Seoul (KR); Byung Kook KIM, Seoul (KR); Hae-Weon LEE, Seoul (KR); Jong Ho LEE, Seoul (KR); Ji-Won SON, Seoul (KR); Kyung Joong YOON, Seoul (KR); Sung Jun CHOI, Seoul (KR); Sung Min CHOI, Seoul (KR)

(73) Assignee: KOREA INSTITUTE OF SCIENCE AND TECHNOLOGY, Seoul (KR)

(21) Appl. No.: 16/021,273

(22) Filed: Jun. 28, 2018

(30) Foreign Application Priority Data
Nov. 20, 2017 (KR) 10-2017-0155046

(51) Int. Cl. B01J 23/00; B01J 23/755; C07C 9/04

(52) U.S. Cl. CPC B01J 23/002; B01J 23/755; C07C 9/04; B01J 35/023; B01J 2523/48; B01J 2523/36; C07C 2523/78; B01J 2523/25

(57) ABSTRACT

A metal/support catalyst for conversion of carbon dioxide to methane contains a metal including a transition metal and a support containing a perovskite-type oxide, on which the metal is supported. The metal/support catalyst for conversion of carbon dioxide to methane is capable of increasing the catalytic activity of the Sabatier reaction by promoting the formation of hydroxide ions and helping the production of formate, which is a reaction intermediate in the conversion of carbon dioxide to methane, without using a precious metal. In addition, it is capable of conducting the reaction stably for a long period of time.

### CROSS-REFERENCE TO RELATED APPLICATIONS

This application claims, under 35 U.S.C. § 119, the priority of Korean Patent Application No. 10-2017-0155046, filed on Nov. 20, 2017, in the Korean Intellectual Property Office, the disclosure of which is incorporated herein by reference in its entirety.

### BACKGROUND

### (a) Technical Field

The present invention relates to a metal/support catalyst for conversion of carbon dioxide (CO₂) to methane (CH₄), more particularly to a metal/support catalyst for conversion of carbon dioxide to methane, which is used for the Sabatier reaction for conversion of carbon dioxide to methane and contains a perovskite (ABO₃)-type oxide capable of conducting protons as a support.

### (b) Background Art

With global environmental issues and global warming problems, demands are increasing on conversion of carbon dioxide to substances useful for our lives. The catalysts that have been used thus far for production of methane from carbon dioxide can be classified into precious metals and transition metal catalysts. The precious metal catalysts represented by ruthenium (Ru) are more superior in performance but are disadvantageous in that they are expensive. Although the transition metal catalysts represented by nickel (Ni) are economically advantageous, they exhibit relatively lower performance and carbon deposition after long-term use.

The performance of many metal/support catalysts developed thus far is largely affected by the metal and does not depend greatly on the characteristics of the support. Although there have been many efforts to alloying the precious metal and the transition metal in order to improve the performance of conversion of carbon dioxide to methane, i.e., the Sabatier reaction, few attempts have been made about changing the support. The representative supports that have been used in the reported experiments are single-component metal oxides such as cerium oxide (CeO₂) and aluminum oxide (Al₂O₃). Cerium oxide shows higher reactivity due to the oxidation-reduction catalytic performance of the material itself but is relatively costly. In contrast, aluminum oxide is relatively inexpensive and exhibits stable structure formation with most metals as well as stable performance, but it shows relatively low performance as compared to cerium oxide.

In the metal/support catalyst, the support constitutes 90% or more of its weight. Therefore, the present metal/support catalyst system, the performance of which depends only on the metal, is of low efficiency. In addition, because high conversion rate and long-term stability are dependent not only on the performance of the precious metal catalyst but also on the characteristics of the support, improvement in the support is necessary.

In the related art, the Sabatier reaction is conducted using a catalyst in which a metal component such as ruthenium or nickel is supported on a single-component oxide such as cerium oxide, aluminum oxide, silicon oxide (SiO₂), titanium oxide (TiO₂), magnesium peroxide (MgO₂), zinc oxide (ZnO), lanthanum oxide (La₂O₃) and yttrium oxide (Y₂O₃).

The present invention provides a metal/support catalyst for conversion of carbon dioxide to methane, which uses a perovskite (ABO₃)-type oxide capable of conducting protons as a support.

### SUMMARY

The present invention is directed to providing a metal/support catalyst for conversion of carbon dioxide to methane capable of increasing the catalytic activity of the Sabatier reaction by promoting the formation of hydroxide ions and helping the production of formate, which is a reaction intermediate in the conversion of carbon dioxide to methane, without using a precious metal and is capable of conducting the reaction for a long period of time.

The metal/support catalyst for conversion of carbon dioxide to methane according to an exemplary embodiment of the present invention contains a metal including a transition metal; and a support containing a perovskite-type oxide, on which the metal is supported.

The support may conduct protons during conversion of carbon dioxide to methane.

The metal may not contain a metal selected from a group consisting of Ru, Rh, Pd, Ag, Ir, Pt and Au.

The metal may contain at least one of Ni, Ti, V, Cr, Mn, Fe, Co, Cu and Zn.

The support may contain at least one of barium zirconate (BaZr₁₋ₓMₓO_{3-δ}), barium cerate (BaCe₁₋ₓMₓO_{3-δ}), strontium zirconate (SrZr₁₋ₓMₓO_{3-δ}), strontium cerate (SrCe₁₋ₓMₓO_{3-δ}), barium zirconate-barium cerate (BaZr_{1-x-y}Ce_{y}MₓO_{3-δ}) and strontium zirconate-strontium cerate (SrZr_{1-x-y}Ce_{y}MₓO_{3-δ}), wherein M is at least one of yttrium (Y), neodymium (Nd), samarium (Sm), ytterbium (Yb), indium (In), europium (Eu) and gadolinium (Gd), 0<x+y<1, 0<x<0.3, 0<y<0.9, and 0<δ<1.

The metal/support catalyst for conversion of carbon dioxide to methane may be used for the Sabatier reaction for conversion of carbon dioxide to methane.

The metal/support catalyst for conversion of carbon dioxide to methane may have a reaction temperature of 300-600° C.

The metal/support catalyst for conversion of carbon dioxide to methane may be in the form of a powder and the powder may have a diameter of 1-50 nanometers (nm).

The metal/support catalyst for conversion of carbon dioxide to methane according to an exemplary embodiment of the present invention is capable of increasing the catalytic activity of the Sabatier reaction by promoting the formation of hydroxide ions and helping the production of formate which is a reaction intermediate in the conversion of carbon dioxide to methane without using a precious metal. In addition, it is capable of conducting the reaction stably for a long period of time.

### BRIEF DESCRIPTION OF DRAWINGS

The patent or application file contains at least one color drawing. Copies of this patent or patent application publication with color drawing will be provided by the USPTO upon request and payment of the necessary fee.

FIG. 1A schematically shows the cross section of a metal/support catalyst for conversion of carbon dioxide to methane according to an exemplary embodiment of the present invention.

FIG. 1B schematically describes a mechanism whereby a metal binds to a support in a metal/support catalyst for conversion of carbon dioxide to methane according to an exemplary embodiment of the present invention.

FIG. 2 shows a result of analyzing the microstructure and elemental distribution of a Ni/BaZr_{0.85}Y_{0.15}O_{3-δ} catalyst in the form of a powder prepared in Example 1 by TEM-EDS.

FIG. 3 shows the carbon dioxide conversion rate, methane yield and methane selectivity of catalysts prepared in Example 1, Comparative Example 1 and Comparative Example 2 depending on reaction temperature, determined by gas chromatography.

FIG. 4 shows the carbon dioxide conversion rate, methane yield and methane selectivity of catalysts prepared in Example 1 and Comparative Example 1 depending on reaction time, determined by conducting gas chromatography once a hour while conducting reaction at 400° C. for 150 hours.

FIG. 5 shows a result of conducting XPS analysis for a Ni/BaZr_{0.85}Y_{0.15}O_{3-δ} catalyst in the form of a powder prepared in Example 1, before and after conducting conversion of carbon dioxide to methane at 300-600° C.

### DETAILED DESCRIPTION

The objectives, other objectives, features and advantages of the present invention will be easily understood through the following detailed description of specific exemplary embodiments and the attached drawings. However, the present invention is not limited to the exemplary embodiments and may be embodied in other forms. On the contrary, the exemplary embodiments are provided so that the disclosure of the present invention is completely and fully understood by those of ordinary skill.

In the attached drawings, like numerals are used to represent like elements. In the drawings, the dimensions of the elements are magnified for easier understanding of the present invention. Although the terms first, second, etc. may be used to describe various elements, these elements should not be limited by the terms. The terms are used only to distinguish one element from another. For example, a first element can be termed a second element and, similarly, a second element can be termed a first element, without departing from the scope of the present invention. A singular expression includes a plural expression unless the context clearly indicates otherwise.

In the present disclosure, the terms such as “include”, “contain”, “have”, etc. should be understood as designating that features, numbers, steps, operations, elements, parts or combinations thereof exist and not as precluding the existence of or the possibility of adding one or more other features, numbers, steps, operations, elements, parts or combinations thereof in advance. In addition, when an element such as a layer, a film, a region, a substrate, etc. is referred to as being “on” another element, it can be “directly on” the another element or an intervening element may also be present. Likewise, when an element such as a layer, a film, a region, a substrate, etc. is referred to as being “under” another element, it can be “directly under” the another element or an intervening element may also be present.

Unless specified otherwise, all the numbers, values and/or expressions representing the amount of components, reaction conditions, polymer compositions or mixtures are approximations reflecting various uncertainties of measurement occurring in obtaining those values and should be understood to be modified by “about”. Also, unless specified otherwise, all the numerical ranges disclosed in the present invention are continuous and include all the values from the minimum values to the maximum values included in the ranges. In addition, when the ranges indicated integers, all the integers from the minimum values to the maximum values included in the ranges are included unless specified otherwise.

The ranges of variables described in the present invention are to be understood to include all the values within the specified end points of the ranges. For example, a range of “5-10” is to be understood to include not only the values 5, 6, 7, 8, 9 and 10 but also any values within subranges such as 6-10, 7-10, 6-9, 7-9, etc. and to include any values between appropriate integers in the specified ranges such as 5.5, 6.5, 7.5, 5.5-8.5, 6.5-9, etc. In addition, for example, a range of “10-30%” is to be understood to include not only the integers 10%, 11%, 12%, 13%, . . . , 30% but also any values within subranges such as 10%-15%, 12%-18%, 20%-30%, etc. and to include any values between appropriate integers in the specified ranges such as 10.5%, 15.5%, 25.5%, etc.

Hereinafter, a metal/support catalyst for conversion of carbon dioxide to methane according to an exemplary embodiment of the present invention is described. In the present invention, the “metal/support catalyst” may mean a catalyst in which a metal is supported on a support.

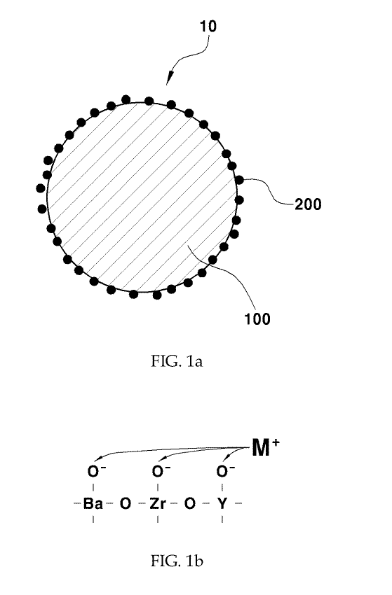

FIG. 1A schematically shows the cross section of a metal/support catalyst 10 for conversion of carbon dioxide to methane according to an exemplary embodiment of the present invention.

Referring to FIG. 1A, the metal/support catalyst 10 for conversion of carbon dioxide to methane according to an exemplary embodiment of the present invention contains a metal 200 and a support 100.

The metal 200 may be supported to form at least one of a nitrate, an acetate, a sulfate and a halide. The metal 200 may be a single metal 200. However, without being limited thereto, the metal 200 may be an alloy of two or more metals.

The metal 200 contains a transition metal. For example, the metal 200 may contain at least one of Ni, Ti, V, Cr, Mn, Fe, Co, Cu and Zn.

The metal 200 does not contain a precious metal. For example, the metal 200 does not contain a metal selected from a group consisting of Ru, Rh, Pd, Ag, Ir, Pt and Au.

The metal 200 is supported on the support 100. The support 100 contains a perovskite (ABO₃)-type oxide which is not a single-component metal oxide (e.g., AO₃-type oxide).

For example, the support 100 may contain at least one of the following compounds in which the site occupied by cerium or zirconium (e.g., the site A in AO₃) is replaced by another element M. For example, the support 100 contains at least one of barium zirconate (BaZr₁₋ₓMₓO_{3-δ}), barium cerate (BaCe₁₋ₓMₓO_{3-δ}), strontium zirconate (SrZr₁₋ₓMₓO_{3-δ}), strontium cerate (SrCe₁₋ₓMₓO_{3-δ}), barium zirconate-barium cerate (BaZr_{1-x-y}Ce_{y}MₓO_{3-δ}) and strontium zirconate-strontium cerate (SrZr_{1-x-y}Ce_{y}MₓO_{3-δ}), wherein M includes at least one of yttrium (Y), neodymium (Nd), samarium (Sm), ytterbium (Yb), indium (In), europium (Eu) and gadolinium (Gd), 0<x+y<1, 0<x<0.3, 0<y<0.9 and 0<δ<1.

The support 100 may contain either a single material or a mixture of two or more materials. The support 100 may be a proton conductor. The proton conductor may conduct protons by forming hydroxide ions under a high-temperature hydrogen or steam atmosphere and, therefore, may promote the conversion of carbon dioxide to methane. More specifically, the metal/support catalyst 10 for conversion of carbon dioxide to methane according to an exemplary embodiment of the present invention may promote the formation of hydroxide ions by using the proton conductor as the support 100 and, therefore, may promote the production of formate (HCOO⁻) which is a reaction intermediate in the conversion of carbon dioxide to methane.

FIG. 1B schematically describes a mechanism whereby the metal binds to the support in the metal/support catalyst for conversion of carbon dioxide to methane according to an exemplary embodiment of the present invention.

For example, referring to FIG. 1A and FIG. 1B, the support 100 may be prepared into a proton conductor by adjusting pH to the point of zero charge or higher. The proton conductor may have a negative surface charge. For example, the support 100 may be a proton conductor to which a metal cation may be bound.

The metal/support catalyst 10 for conversion of carbon dioxide to methane may have a reaction temperature of 300-600° C. If the reaction temperature is below 300° C., it may not function as a catalyst during conversion of carbon dioxide to methane because the metal 200 is not activated sufficiently. And, a reaction temperature exceeding 600° C. is not suitable for methane production due to thermodynamically low reaction affinity.

The metal/support catalyst 10 for conversion of carbon dioxide to methane may be used for the Sabatier reaction for conversion of carbon dioxide to methane. More specifically, the metal/support catalyst 10 for conversion of carbon dioxide to methane according to an exemplary embodiment of the present invention, wherein a proton conductor is used as the support 100, may promote the formation of hydroxide ions and, accordingly, may promote the formation of formate (HCOO⁻) which is a reaction intermediate during the conversion of carbon dioxide to methane.

The metal/support catalyst 10 for conversion of carbon dioxide to methane may be in the form of a powder. The powder may have a diameter of 1-50 nanometers (nm). A catalyst in the form of a powder having the diameter of the above range may be prepared by adjusting pH using urea. If the diameter of the powder is smaller than 1 nanometer, it may not function as a catalyst because the characteristics of the metal are not exerted. And, if the diameter of the powder exceeds 50 nanometers, reaction rate may decrease because of a small surface area.

For example, the metal/support catalyst for conversion of carbon dioxide to methane according to an exemplary embodiment of the present invention may be used in at least one of a reactor using conversion of carbon dioxide to methane, a fuel cell using a proton conductor, an electrochemical device using electrochemical and thermochemical reaction of carbon dioxide, an electrolysis system of hydrogen compounds, a hydrogen sensor, a hydrogen device used in decomposition of hydrogen gas and a ceramic hydrogen pump.

The metal/support catalyst for conversion of carbon dioxide to methane according to an exemplary embodiment of the present invention can increase the catalytic activity of the Sabatier reaction by promoting the formation of hydroxide ions and helping the production of formate, which is a reaction intermediate in conversion of carbon dioxide to methane, although it does not contain a precious metal. In addition, the reaction can be conducted stably for a long period of time.

The present invention will be described in more detail through examples. The following examples are for illustrative purposes only and it will be apparent to those skilled in the art that the scope of this invention is not limited by the examples.

### EXAMPLE 1

As a support, 1 g of barium zirconate (BaZr_{0.85}Y_{0.15}O_{3-δ}) substituted with 15 mol % yttrium was added to 20 mL of water and stirred at 500 rpm using a magnetic bar. 5 wt % of nickel nitrate based on the total weight of a catalyst was dissolved in 10 mL of water. The nickel nitrate aqueous solution was added to the support aqueous solution being stirred and the temperature was raised to 90° C. The pH of the solution was increased by adding 0.3 g of urea. After conducting reaction sufficiently for 4 hours, the solution was cooled rapidly using liquid nitrogen. The cooled powder was freeze-dried for about 12 hours. The dried powder was put in an aluminum oxide crucible, sintered at 600° C. for 3 hours and reduced at 600° C. for 2 hours under a 4% H₂ atmosphere to obtain a Ni/BaZr_{0.85}Y_{0.15}O_{3-δ} catalyst in the form of a powder. In this example 3—δ may be 2.925, although not being limited thereto.

### COMPARATIVE EXAMPLE 1

As a support, 1 g of aluminum oxide (Al₂O₃) was added to 20 mL of water and stirred at 500 rpm using a magnetic bar. 5 wt % of nickel nitrate based on the total weight of a catalyst was dissolved in 10 mL of water. The nickel nitrate aqueous solution was added to the support aqueous solution being stirred and the temperature was raised to 90° C. The pH of the solution was increased by adding 0.3 g of urea. After conducting reaction sufficiently for 4 hours, the solution was cooled rapidly using liquid nitrogen. The cooled powder was freeze-dried for about 12 hours. The dried powder was put in an aluminum oxide crucible, sintered at 400° C. for 3 hours and reduced at 600° C. for 2 hours under a 4% H₂ atmosphere to obtain a Ni/Al₂O₃ catalyst in the form of a powder.

### COMPARATIVE EXAMPLE 2

A Ru/Al₂O₃ catalyst in the form of a powder was prepared in the same manner as in Comparative Example 1 except that a ruthenium nitrosyl nitrate solution was used as a metal precursor in order to support Ru metal.

### TEST EXAMPLE 1

### Analysis of Microstructure and Structural Stability of Synthesized Catalyst by TEM

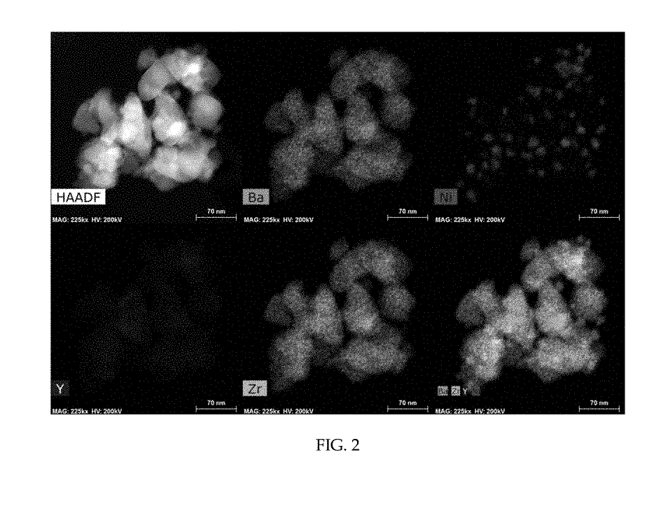

In order to investigate the microstructure and structural stability of the catalyst in the form of a powder obtained in Example 1, images and elemental mapping data obtained by transmission electron microscopy (TEM) and energy-dispersive X-ray spectroscopy (EDS) were analyzed. The result is shown in FIG. 2.

Referring to FIG. 2, it was confirmed that a stable phase is maintained because all the elements constituting the support were uniformly distributed. The particle size of the supported Ni metal was about 13.9 (±2.9) nm.

### TEST EXAMPLE 2

### Gas Chromatography Analysis of Catalyst Using Proton Conductor as Support

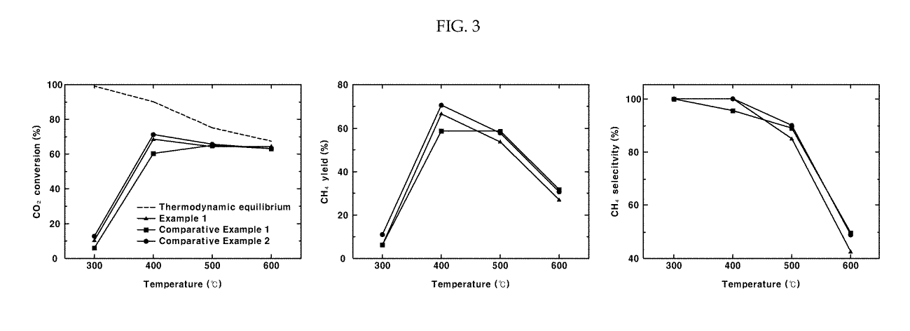

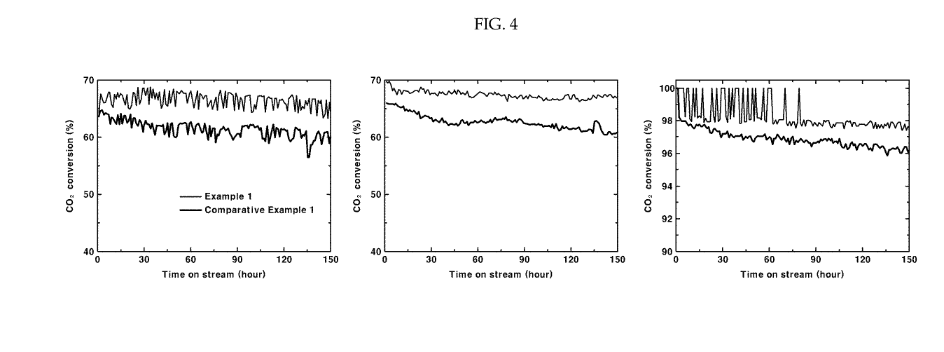

For comparison of the performance of the synthesized catalysts, methane conversion reaction was conducted using the catalyst prepared in Example 1 using a proton conductor as a support and gas chromatography analysis was carried out. The reaction was conducted by placing the catalyst in a tube-type quartz reactor operating under atmospheric pressure. The reactor was placed again in a tube-type electrical furnace for control of reaction temperature. Carbon dioxide (CO₂) conversion rate, methane (CH₄) yield and methane (CH₄) selectivity) were compared with those of the single metal catalysts (Comparative Examples 1 and 2). The result is shown in FIG. 3 and FIG. 4.

Referring to FIG. 3, the catalyst of Example 1 prepared at 400° C. showed about 8% increased carbon dioxide conversion rate as compared to Comparative Example 1, which was about 3% lower as compared to Comparative Example 2 wherein the precious metal was used. Also in terms of methane selectivity, unlike Comparative Example 1 which showed a methane selectivity of about 95% at 400° C., Example 1 showed a methane selectivity of about 100% comparable to that of Comparative Example 2 wherein the precious metal was used.

FIG. 4 shows the carbon dioxide conversion rate, methane yield and methane selectivity of the catalysts prepared in Example 1 and Comparative Example 1 depending on reaction time, determined by conducting gas chromatography once a hour while conducting reaction at 400° C. for 150 hours. Referring to FIG. 4, Example 1 showed about 4% decreased methane yield after 150 hours, whereas Comparative Example 1 showed about 7% of decrease. Example 1 showed about 43% improved heat resistance in the long-term stability test for 150 hours as compared to Comparative Example 1. Also in terms of selectivity, Example 1 showed a methane selectivity of 100% for up to a maximum of 79 hours and about 97.5% after 150 hours. In contrast, Comparative Example 1 did not show 100% of methane selectivity except for the first 1 hour. The methane selectivity was decreased gradually with time to 96.3% after 150 hours.

### TEST EXAMPLE 3

### Analysis of Hydroxide Ions (OH⁻) on Catalyst Surface by XPS

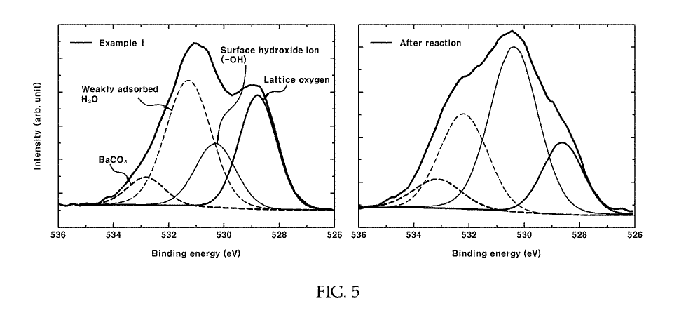

After conducting conversion of carbon dioxide to methane using the catalyst in the form of a powder obtained in Example 1, O 1 s peaks were analyzed by X-ray photoelectron spectroscopy (XPS) to detect hydroxide ions on the surface of the catalyst. The result is compared with that before conducting the reaction in FIG. 5.

FIG. 5 shows the result of conducting XPS analysis on the Ni/BaZr_{0.85}Y_{0.15}O_{3-δ} catalyst in the form of a powder prepared in Example 1, before and after conducting conversion of carbon dioxide to methane at 300-600° C. Referring to FIG. 5, the presence of hydroxide ions were confirmed through O 1 s peak analysis, and when compared with the non-reacted powder, it was confirmed that the hydroxide ion peak at 530.3 eV was increased after conducting the reaction. Accordingly, it was confirmed that the presence of hydroxide ions formed on the support powder contributes to the improvement in reaction yield and selectivity under the Sabatier reaction condition.

The present invention has been described in detail with reference to specific embodiments thereof. However, it will be appreciated by those skilled in the art that various changes and modifications may be made in these embodiments without departing from the principles and spirit of the invention, the scope of which is defined in the appended claims and their equivalents.

1. A metal/support catalyst for conversion of carbon dioxide to methane, comprising:
a support comprising a perovskite (ABO₃) oxide; and
a metal embedded on the support,
wherein the metal comprises a transition metal.

2. The metal/support catalyst for conversion of carbon dioxide to methane according to claim 1, wherein the support conducts protons during conversion of carbon dioxide to methane.

3. The metal/support catalyst for conversion of carbon dioxide to methane according to claim 1, wherein the metal does not comprise a metal selected from a group consisting of Ru, Rh, Pd, Ag, Ir, Pt and Au.

4. The metal/support catalyst for conversion of carbon dioxide to methane according to claim 1, wherein the metal comprises at least one of Ni, Ti, V, Cr, Mn, Fe, Co, Cu and Zn.

5. The metal/support catalyst for conversion of carbon dioxide to methane according to claim 1, wherein the support comprises at least one of barium zirconate (BaZr₁₋ₓMₓO_{3-δ}), barium cerate (BaCe₁₋ₓMₓO_{3-δ}), strontium zirconate (SrZr₁₋ₓMₓO_{3-δ}), strontium cerate (SrCe₁₋ₓMₓO_{3-δ}), barium zirconate-barium cerate (BaZr_{1-x-y}Ce_{y}MₓO_{3-δ}) and strontium zirconate-strontium cerate (SrZr_{1-x-y}Ce_{y}MₓO_{3-δ}), wherein
M is at least one of yttrium (Y), neodymium (Nd), samarium (Sm), ytterbium (Yb), indium (In), europium (Eu) and gadolinium (Gd),
0<x+y<1,
0<x<0.3,
0<y<0.9, and
0<δ<1.

6. The metal/support catalyst for conversion of carbon dioxide to methane according to claim 1, wherein the metal/support catalyst for conversion of carbon dioxide to methane is used for the Sabatier reaction for conversion of carbon dioxide to methane.

7. The metal/support catalyst for conversion of carbon dioxide to methane according to claim 1, wherein the metal/support catalyst for conversion of carbon dioxide to methane has a reaction temperature of 300-600° C.

8. The metal/support catalyst for conversion of carbon dioxide to methane according to claim 1, wherein the metal/support catalyst for conversion of carbon dioxide to methane is in the form of a powder and the powder has a diameter of 1-50 nanometers (nm).
